(19) United States
(12) Patent Application Publication
(10) Pub. No.: US 2013/0237964 A1
(43) Pub. Date: Sep. 12, 2013

(54) NON-INVASIVE URINE DIRECTIONAL DEVICE, URINE COLLECTION SYSTEM, AND KIT

(71) Applicant: JOHN KICOS, Cornelius, NC (US)

(72) Inventor: JOHN KICOS, Cornelius, NC (US)

(21) Appl. No.: 13/780,130

(22) Filed: Feb. 28, 2013

Related U.S. Application Data
(60) Provisional application No. 61/634,855, filed on Mar. 7, 2012.

(51) Int. Cl. A61F 5/44; A61M 25/00

(52) U.S. Cl. CPC A61F 5/4408; A61M 25/0017
USPC 604/544; 604/347

(57) ABSTRACT

A non-invasive urine directional device, urine collection system, and kit are provided for a physically active user. The urine directional device includes a form-fitting undergarment or a band fastened to an open top, non-invasive receiving body in fluid communication with a drainage element. The urine collection system includes a urine directional device and a urine disposal assembly having urine collection bags. The kit includes a reusable urine directional device and a reusable wash bag.

### TECHNICAL FIELD

This invention generally relates to a non-invasive urine directional device, urine collection system, and kit useful for physically active females and males.

### BACKGROUND

Incontinence and over-active bladders often present a challenge to men and women who want to remain active. Further, some physical and work activities are performed where restroom facilities are not readily available. For example, physical activities such as hunting, endurance competitive sports, cold-weather outdoor sports, and the like, can present challenges that make urine elimination inconvenient, particularly for women. Work settings, such as the military service, may also not be conducive to a typical means of urine elimination. Women, in particular, often purposely dehydrate themselves to avoid the need to urinate in these situations, which can present health issues such as urinary tract infections and dehydration.

Although many urinary collection devices exist, the devices are generally not designed for physically activity users. Often, the devices are invasive, complex and uncomfortable. Invasive devices may place the user at a heightened risk for urinary infections. Further, the complexity of invasive design increases the cost of manufacture. Disposable diapers, incontinence pads or undergarments with absorbent features provide alternatives to the complex devices but present other undesirable issues because there is typically no means to convey the urine away from the user. Further, the disposable aspect of this group of devices is not environmentally friendly as reuse is prohibited.

There remains a need to provide men and women with comfortable, non-invasive urine collection devices that permit physical activity. This disclosure describes an improvement over these prior art technologies.

### SUMMARY

In one embodiment, in accordance with the principles of the present disclosure, a urine directional device is provided. The urine directional device includes a form-fitting undergarment; a non-invasive receiving body; and a drainage element. The form-fitting undergarment has a pant shape with a first defined leg portion, a second defined leg portion, and an open crotch portion. The first defined leg portion and the second defined leg portion are configured to extend to at least an upper thigh region of a user's legs. The non-invasive receiving body is configured to receive and direct a urine flow away from a user's body. The receiving body has an open-top proximal end that tapers to an open outlet at a distal end. The proximal end of the receiving body is fastened to the open crotch portion of the form-fitting undergarment around a circumference of the open crotch portion. The distal end of the receiving body is fastened to the drainage element. The drainage element is in fluid communication with the receiving body and configured to extend a urine flow away from the user's body. The form-fitting undergarment holds the non-invasive receiving body snugly to permit the user to participate in physical activity. Another advantage of the device is that it is reusable.

In accordance with the principles of the present disclosure, another embodiment provides a non-invasive urine directional device having a band rather than a form-fitting undergarment to which a receiving body and drainage element is fastened. The band is configured with a non-invasive receiving body having a funnel shape that tapers from an open top at a proximal end to an open outlet at a distal end. The band includes at least a front strap and a back strap. The front strap has a proximal end and a distal end, the proximal end of the front strap fastened to a front portion of the band and a front connector portion located on a front section of the receiving body. The back strap has an inverted Y-shaped configuration with a single proximal portion that divides into a first distal end and a second distal end. The single proximal portion of the back strap is fastened to a back portion of the band. The first distal end is fastened to a first back connector portion located on a back section of the receiving body. The second distal end is fastened to a second back connector portion located on the back section of the receiving body. The drainage element is configured to extend a urine flow away from a user's body with an open outlet of the receiving body fastened to the drainage element. The drainage element is in fluid communication with the receiving body. The band holds the non-invasive receiving body in place when disposed upon a user, and the device is reusable.

In another embodiment, in accordance with the principles of the present disclosure, a non-invasive urine collection system is provided. The system comprises a urine directional device; and urine disposal assembly. The urine directional device includes a form-fitting undergarment or a band; a non-invasive receiving body; and a drainage element. The urine disposal assembly includes a urine collection bag in fluid communication with the receiving body and drainage element of the urine directional device. A fastener is configured to detachably connect the drainage element of the urine directional device to the urine disposal assembly.

In another embodiment, a kit is provided. The kit comprises a reusable urine directional device configured to extend a urine flow away from a user's body; a urine disposal assembly; and a reusable wash bag configured to hold the urine directional device during a cleansing process. The kit provides an environmentally friendly alternative to disposable urine collection systems.

### BRIEF DESCRIPTION OF THE DRAWINGS

The present disclosure will become more readily apparent from the specific description accompanied by the following drawings, in which:

FIG. 1 is a front perspective view of a urine directional device in accordance with the principles of the present disclosure;

FIG. 2 is a top plan view of a component of the urine directional device shown in FIG. 1;

FIG. 3 is a side plan view of a component of the urine directional device of FIG. 1;

FIG. 4 is a front perspective view of the urine directional device disposed upon a user;

FIG. 5 is a front perspective view of a urine collection system;

FIG. 6 is a front perspective view of the urine collection system disposed upon a user;

FIG. 7 is a front perspective view of a urine directional device in accordance with the principles of the disclosure; and

FIG. 8 is a front perspective view of a kit in accordance with the principles of the disclosure.

Like reference numerals indicate similar parts throughout the figures.

### DETAILED DESCRIPTION

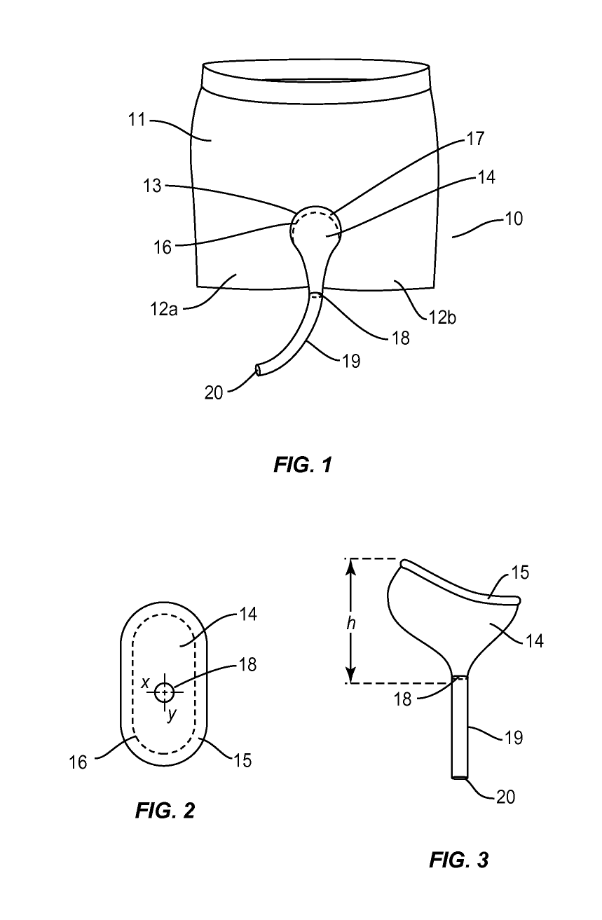

The present disclosure can be understood more readily by reference to the following detailed description of the disclosure taken in connection with the accompanying drawing FIGS. 1-8, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges can be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, distal and proximal, are for illustrative purposes only and can be varied within the scope of the disclosure. For example, the references “upper” and “lower” are relative and used only in the context to the other, and are not necessarily “superior” and “inferior”.

FIG. 1 illustrates a urine directional device 10 in accordance with an embodiment of the invention. As shown in FIG. 1, a form-fitting undergarment 11 has a pant shape with a first leg portion 12a and a second leg portion 12b and an open crotch portion 13. An open top of a receiving body 14 is fitted around a circumference of the open crotch portion 13. As shown in FIGS. 2 and 3, the receiving body 14 can include an optional outer ridge 15 around the circumference of the open top of the receiving body 14. The circumference of the open top of the receiving body 14 is fastened to the circumference of the open crotch portion 13 with a fastening means 16 to form a gap-free seal between the form-fitting undergarment 11 and the receiving body 14. The receiving body 14 is configured to be held snugly in place within the form-fitting undergarment 11 to permit physical activity of the user.

The receiving body 14 is a hollow, molded piece configured to direct a urine flow away from a user. In one embodiment, the receiving body 14 is in the form of an open funnel, with the proximal end 17 configured to fit the contours of the female anatomic pelvic structures such that the receiving body 14 is non-invasive and sufficient in proportion to impart the urine directional device 10 substantially leakage proof capabilities during the voiding of urine. In another embodiment, the receiving body 14 is configured to fit the male anatomy such that the fit is non-invasive and sufficient in proportion to impart the urine directional device 10 with leakage proof capabilities during the voiding of urine. As shown in FIGS. 1-3, the proximal end 17 of the receiving body 14 tapers to an open outlet 18 at a distal end of the receiving body. The open outlet 18 is positioned approximately in a center of the receiving body 14 to guide the urine flow away from the user into a drainage element 19, as shown in FIG. 2.

The form-fitting undergarment 11 is manufactured or assembled as a one-piece garment made from a form-fitting material, preferably a 360-degree stretch fabric, capable of moving with the user's body through all ranges of motion. The form-fitting undergarment 11 can be manufactured in different sizes to fit the user comfortably and accommodate physical activity. The form-fitting material contributes to snugly keeping the receiving body 14 in functional placement upon fastening the form-fitting undergarment 11 to the receiving body 14. Materials that can be used for the form-fitting undergarment 11 include nylon, LYCRA®, spandex and other shaper fabrics that conform to the user's body. One such material comprises nylon and spandex, preferably about 90% nylon and 10% spandex. The form-fitting undergarment 11 can include construction elements such as a waistband and trim to provide additional durability to the urine directional device 10.

As shown in FIG. 1, each leg portion 12a, 12b of the form-fitting undergarment extends to at least an upper thigh region of the user's legs. Depending upon the user's preference each leg portion 12a, 12b can also be constructed to extend to a mid-thigh region, a knee region, or an ankle region. Each leg portion 12a, 12b is configured to pull the receiving body 14 into an extended open position when the user adopts a widened stance with legs positioned apart during a urine void such that all of a urine flow is directed into the receiving body 14. For cold weather, outdoor physical activity, such as cross-country skiing, dog sledding, and the like, longer leg portions provide an additional benefit of an extra layer of warmth.

As illustrated in FIG. 2, a vertical y axis length of the receiving body 14 is longer than a horizontal x axis width of the receiving body 14, with the vertical length y fitted into the open crotch portion 13 of the form-fitting undergarment 11 from the front to the back, and the horizontal length x fitted into the open crotch portion 13 of the form-fitting undergarment 11 from side to side. For a female user, the vertical y axis of the receiving body 14 extends out to a length ranging from about 15 cm to about 25 cm, with approximately 21 cm representing the length of the receiving body 14 for an average sized female user. For a female user, the horizontal x axis of the receiving body 14 generally extends out to a width of from about 5 cm to about 15 cm, with approximately 6 cm representing the width of the receiving body 14 for the average sized female user. For a male user, the vertical y axis of the receiving body 14 extends out to a length ranging from about 15 cm to about 25 cm, with approximately 16 cm representing an average length of the receiving body 14. For a male user, the horizontal x axis of the receiving body 14 generally extends out to a width of about from about 10 cm to about 25 cm, with approximately 15 cm representing an average width of the receiving body 14. For an average sized male user, a diameter of the circumference of the open top of the receiving body 14 is approximately 31 cm.

The receiving body 14 is constructed from a material that is comfortable and of sufficient rigidity to provide structure to secure the receiving body 14 in place during different ranges of motion. Materials used for the receiving body 14 can be selected from any number of flexible, waterproof, and/or hydrophobic materials compatible to human skin. Examples of appropriate materials include silicon based materials; FDA approved medical grade polyurethane; and the like. The thickness of the material selected depends upon the user's needs, taking into account, for example, the duration of each wearing, cost constraints, reuse expectations, considerations relating to the fit of outer clothing, and the like. In one embodiment, the material for the receiving body 14 has a thickness ranging from 0.05 to 0.25 cm, more preferably 0.1 to 0.3 cm. FDA approved medical grade polyurethane that can be used for the receiving body 14 has an approximate thickness of 0.15 cm.

The receiving body 14 is configured to be substantially undetectable when disposed underneath outer clothing. The receiving body 14 is proportionally sized in accordance with the size of the user, thus contributing to the receiving body 14 being inconspicuous underneath outer clothing. That is, a user fitting into a small form-fitting undergarment 11 will preferably have a correspondingly small receiving body 14 assembled into the form-fitting undergarment 11. For female users, the receiving body proximal end 17 is configured such that external female genitalia are generally not in direct contact with the receiving body 14. This non-invasive configuration can decrease the problem of vaginitis and also makes the directional device 10 substantially leak proof.

As illustrated in FIG. 3, the receiving body 14 is molded in a boat-like shape, with a height h of the receiving body 14 extending from the proximal end 17 to the open outlet 18 at the distal end. For an average sized female, the height h of the receiving body is approximately 10 cm.

As shown in FIGS. 1-6, a fastener 16 is used to fasten the form-fitting undergarment 11 to the receiving body 14. Any number of fastening mechanisms can be used, including zippers, adhesives, stitching, and the like. In one embodiment, the open crotch region 13 is permanently affixed to the receiving body 14 with the outer edge 15 placed inside the form-fitting undergarment 11 such that the presence of the receiving body 14 is more inconspicuous when covered with clothing. In one embodiment, at least one seam is used as the fastener 16 to permanently fasten the receiving body 14 and form-fitting undergarment 11 around the circumference of the proximal end 17 of the receiving body 14 to form one piece that can be easily cleaned, thus promoting reuse.

The drainage element 19 is configured to provide an unobstructed flow path for the urine to drain away from the user's body. As shown in FIG. 1, the drainage element 19 is a tube. The receiving body 14 and the drainage element 19 can be manufactured as separate molded pieces and thereafter fastened or connected. Alternatively, the receiving body 14 and drainage element 19 can be manufactured as a single molded unit. Generally, materials used for the drainage element 19 should be flexible yet of a sufficient rigidity such that the drainage element 19 will not collapse during physical activity and movement of the user. To impart different structural components, the drainage element 19 can be constructed from more than one material. Preferred materials for the drainage element 19 include silicone based materials, FDA approved medical grade polyurethane, and the like. For ease of manufacture, the same material used for the receiving body 14 can also be used for the drainage element 19. In one embodiment, the material for the receiving body 14 has a thickness ranging from 0.05 to 0.25 cm, more preferably 0.1 to 0.3 cm, most preferably about 0.15 cm.

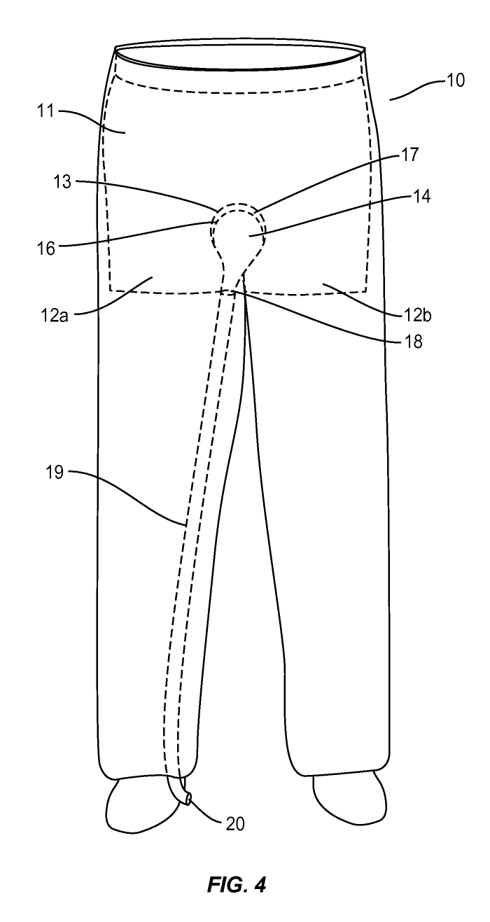

The drainage element 19 includes a hollow tubing that permits fluid communication between the receiving body 14 and the drainage element 19. The drainage element 19 is configured with a length sufficient to guide through a distal end 20 of the drainage element 19 away from the user's body. Preferably, the drainage element 19 is configured to have a length of at least 15 cm. For an average sized female to extend the drainage element to mid-thigh, the length of the drainage element 19 will be approximately 21 cm when measuring from the open outlet 18 of the distal end of the receiving body 14 to the distal end 20 of the drainage element 19 (or approximately 31 cm when measuring from the proximal end 17 of the receiving body 14 to the distal end 20 of the drainage element). The drainage element 19 can be detachably connected to the receiving body 14 such that the user can alternate between different lengths of the drainage element 19 if desired by the user. Alternatively, the drainage element 19 can be permanently affixed to the open outlet 18 of the receiving body 14 using an adhesive, other bonding mechanism or substance, or molding. As shown in FIG. 4, for extreme weather conditions or in certain work conditions, the extension of the drainage element 19 to the user's ankle eliminates the need to remove outer clothing during urine elimination. This is particularly useful for women who have multiple layers of clothing in adverse conditions. For the drainage element 19 configured to extend to the ankle, the length of the drainage element 19 is approximately 90 cm for the average female height. One or more supports can be used to strap or otherwise secure the drainage element 19 to the user's leg or otherwise attach or affix to the user's clothing, if desired by the user. When the directional device 10 is disposed upon a user, as shown in FIG. 4, the user adopts an upright position during urine elimination to avoid leakage.

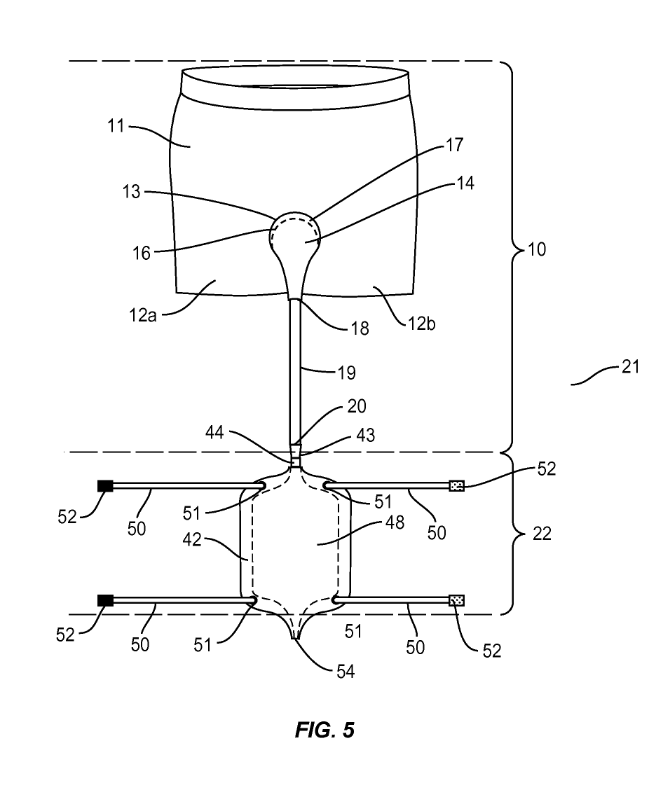

In FIG. 5, a urine collection system 21 is shown where the urine directional device 10 further comprises a urine disposal assembly 22. The urine disposal assembly 22 includes a urine collection bag 42 that is in fluid communication with the receiving body 14 and the drainage element 19 of the urine directional device 10. The distal end 20 of the drainage element 19 is configured for a detachable connection with the urine disposal assembly 22 using a fastener 43. Any suitable fastener mechanism may be used, including a clamp, a flange, a clip, a snap retaining ring, a tie strap, and the like. As shown in FIG. 5, the fastener 43 is a flange having a series of circular ridges. The drainage element 19 is detached from the urine collection bag 42 whenever the user wishes to empty, clean or dispose of the urine collection bag 42. The receiving body 14, drainage element 19, and urine collection bag 42 are all in fluid communication in the urine collection system 21.

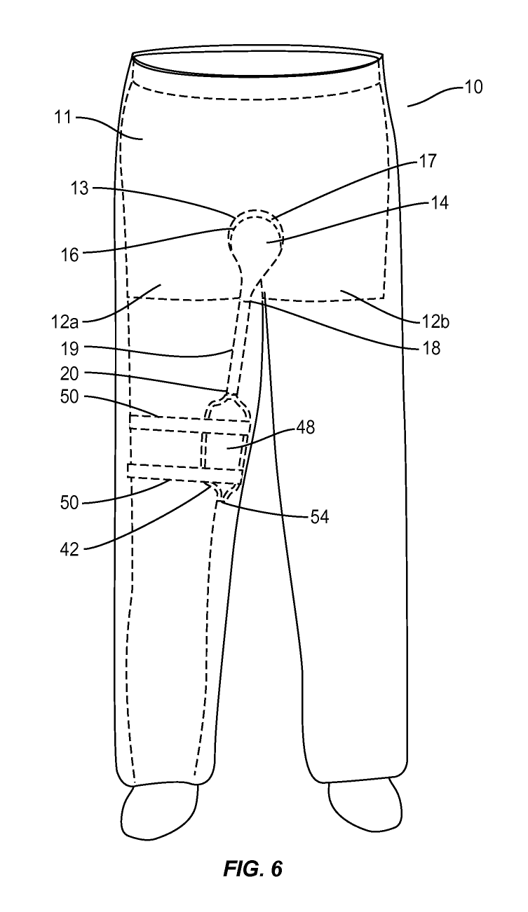

As illustrated in FIG. 5, the fastener 43 is connected to the one-way valve 44 to prevent back-flow from the urine collection bag 42. The urine collection bag 42 is configured with the urine reservoir 48 inside the urine collection bag 42. A system of one or more support elements 50 can be affixed to the urine collection bag 42 through a system of one or more recesses 51 including holes or other such perforations that are outside of the reservoir 48 as shown in FIG. 5. As shown in FIG. 6, the one or more support element(s) 50 can be extended around the circumference of the user's leg and held together with a mating attachment mechanism 52 such as VELCRO® fastening tabs. The urine collection bag 42 can be configured with a drainage piece 54 including a drain port or valve configured with an open and closed position such that the user can empty the urine collection bag 42 after use. Alternatively, the urine collection bag 42 can be disposable and thus require no drainage piece.

As illustrated in FIG. 5, the urine collection bag 42 receives and stores urine collected from the receiving body 14 as drained through the drainage element 19. Any waterproof material compatible with skin can be used for constructing the urine collection bag 42. The size of the urine collection bag 42 is of sufficient proportion to accommodate typical voiding quantities, with different sizes possible depending upon the duration of time that the urine collection system 22 is in use and the degree of physical activity exerted by the user. The bag 42 preferably holds at least 250 ml volume and can be configured for volumes up to 1000 ml or 1500 ml and beyond to extend the time of usage. The shape of the urine collection bag 42 can be selected from any number of commercially available urine bags or specifically configured for the user's needs where the shape can be contoured, straight, wide, slim and long, and the like. In one embodiment, the urine collection bag is transparent to permit the user to observe the level of fluid in the bag reservoir 48. Commercially available urine collection bags 42 include Freedom leg bags by Coloplast-Mentor (Minneapolis, Minn.). The urine collection bag 42 can be located on any desired region of the leg, including the user's upper thigh, lower thigh, calf, or ankle, on the side, back or front region of the leg.

As illustrated in FIG. 6, the urine collection bag 42 can be placed inconspicuously on the user's leg such that an outer garment covers the urine collection system 21. The urine collection bag 42 can optionally include a portion with a comfort backing, such as a soft fabric affixed to the urine collection bag 42. The comfort backing may be laminated, glued, stitched, or otherwise attached to or manufactured as a part of the urine collection bag 42. In one embodiment, the portion of the urine collection bag 42 having the comfort backing includes the back region resting against the user's leg, with the front region of the urine collection bag 42 comprising a transparent material to show an amount of urine present in the bag reservoir 48 at any given time.

As shown in FIG. 6, when disposed upon a user, the urine collection system 21 is configured to provide the user with a hand's free portable and inconspicuous means of urine elimination which can be of particular benefit to women. The optimal position for a user of the urine collection system 21 is an upright stance.

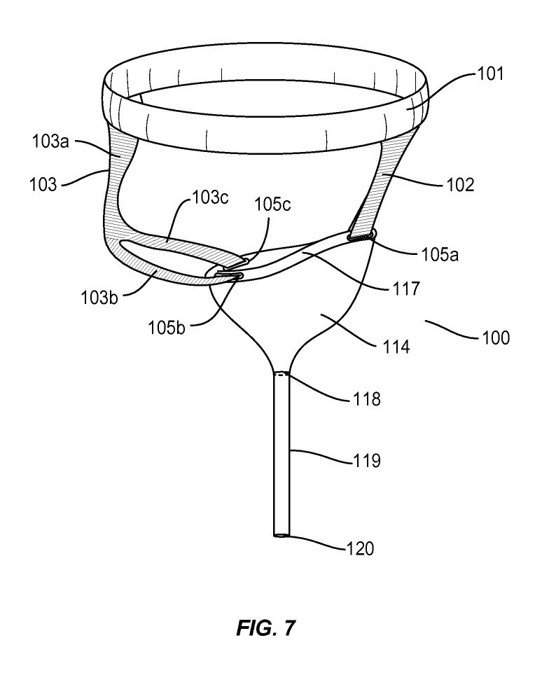

FIG. 7 illustrates a non-invasive urine directional device 100 that includes a band 101 configured with a non-invasive receiving body 114; and a drainage element 119. The band 101 replaces the form-fitting undergarment 11 shown in the configuration of FIG. 1 as the support for holding the receiving body 114 in place when disposed on the user. As shown in FIG. 7, the receiving body 114 has an open top with at least three connection portions 105a, 105b, 105c. The receiving body 114 is configured to have a front section and a back section. The band 101 is made of an elastic material or other stretchable material, with sizing in a small, medium, large, and so on. The band 101 includes at least a front strap 102 and a back strap 103. The front strap 102 has a proximal end and a distal end. The proximal end of the front strap 102 is fastened to a front portion of the band 101. The distal end of the front strap 102 is fastened to a front connection portion 105a located on the front section of the receiving body 114. The back strap 103 has an inverted Y-shaped configuration with a single proximal portion 103a that is fastened to a back portion of the band 101. The single proximal portion 103a of the back strap 103 divides into a first distal end 103b and a second distal end 103c. The first distal end 103b is fastened to a first back connector portion 105b located on the back section of the receiving body 114. The second distal end 103b is fastened to a second back connector portion 105c located on the back section of the receiving body 114. The front strap 102 and the back strap 103 can be detachably fastened to the band 101 or permanently fastened to the band 101. Similarly, the front strap 102 and the back strap 103 can be detachably fastened to the receiving body 114 or permanently fastened to the receiving body 114. For the detachable embodiments, the straps 102, 103 can be mattingly fastened using hooks, snaps, VELCRO® strips and the like. A proximal end 117 of the receiving body 114 tapers in a funnel shape to an open outlet 118 at a distal end of the receiving body 114. The receiving body 114 is configured to fit the male anatomy or the female anatomy in a manner sufficient to prevent any leakage of urine during use. Materials and dimensions of the receiving body 114 are the same as described for the receiving body 14 shown in FIG. 1. A drainage element 119 is fastened to the open outlet 118 of the receiving body 114 or molded as a single, congruent piece with receiving body 114. The drainage element 119 is in fluid communication with the receiving body 114, with the drainage element 119 configured to extend a urine flow away from a user's body. The distal end 120 of the drainage element 119 provides an outlet for the urine.

The urine directional device 100 shown in FIG. 7 is configured such that the user can void in a sitting position in addition to a standing position. This sitting position is particularly useful for a male user as the device 100 provides an alternative to invasive catheters. In one configuration in accordance with the principles of the disclosure, the urine directional device 100, as shown in FIG. 7, is configured to be utilized when a user is in a sitting position, raising one leg slightly during a voiding to further ensure against leakage.

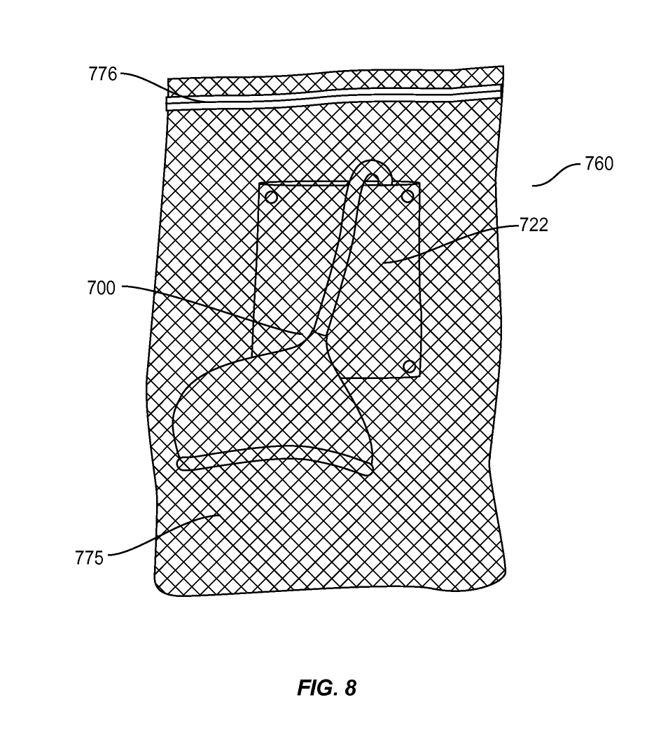

In accordance with the principles of the disclosure, a kit 760 is also provided. As shown in FIG. 8, the kit 760 includes the reusable urine directional device 700 (10, 100) and configured to extend a urine flow away from a user's body; the urine disposal assembly 722 (22); and a reusable wash bag 775. The reusable wash bag 775 is configured to hold at least the urine directional device 700 (10, 100) during a cleansing process. The reusable wash bag 775 can be made from any number of fabrics or materials including a washable net fabric with a zipper 776 or other type of closure. Optionally, the kit can be assembled to include only the reusable wash bag 775 and the urine directional device 700.

It will be understood that various modifications can be made to embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A urine directional device including:
(a) a form-fitting undergarment having a pant shape with a first defined leg portion, a second defined leg portion, and an open crotch portion, the first defined leg portion and the second defined leg portion configured to extend to at least an upper region of a user's thighs;
(b) a receiving body configured to be non-invasive and to receive and direct a urine flow away from a user's body, the receiving body having an open-top proximal end that tapers to a distal end, the proximal end fastened to the open crotch portion of the form-fitting undergarment around a circumference of the open crotch portion; and
(c) a drainage element in fluid communication with the receiving body and configured to extend a urine flow away from a user's body, the drainage element fastened to the distal end of the receiving body.

2. The device according to claim 1 wherein said form-fitting undergarment is made from a shaper material.

3. The device according to claim 2 wherein said form-fitting undergarment is constructed from a material comprising 10% spandex and 90% nylon.

4. The device according to claim 3 wherein the form-fitting undergarment is a 360-degree stretch material.

5. The device according to claim 4 wherein the receiving body is constructed from a material selected from the group consisting of a silicone based material, a polyurethane, or a combination thereof.

6. The device according to claim 5 wherein drainage element constructed from a material selected from the group consisting of a silicone based material, a polyurethane, or a combination thereof.

7. The device according to claim 6 wherein the receiving body and the drainage element are constructed from the same material.

8. The device according to claim 7 wherein the receiving body is molded in a boat-like shape.

9. The device according to claim 8 wherein a user is a female and the open-top of the receiving body is configured to further open when the female stands with legs held apart to void urine.

10. The device according to claim 9 wherein said form-fitting undergarment is permanently fastened to said receiving body by stitching.

11. The device according to claim 10 wherein the drainage element is a tube of a length of approximately 15 cm.

12. The device according to claim 10 wherein the drainage element is a tube of a length of approximately 90 cm.

13. The device according to claim 1 further comprising a urine disposal assembly comprising a urine collection bag in fluid communication with the drainage element and the receiving body of the urine directional device, the urine disposal assembly including a fastener configured to detachably connect the urine collection bag to the drainage element.

14. The device according to claim 13 wherein at least a portion of the urine collection bag includes a comfort fabric backing.

15. The device according to claim 14 wherein the drainage element is constructed from polyurethane.

16. A non-invasive urine directional device including:
(a) a band configured with a non-invasive receiving body, the receiving body having a funnel shape that tapers from an open top at a proximal end to an open outlet at a distal end, the band including at least a front strap and a back strap, the front strap having a proximal end and a distal end, the proximal end of the front strap fastened to a front portion of the band and a front connector portion located on a front section of the receiving body, the back strap having an inverted Y-shaped configuration with a single proximal portion that divides into a first distal end and a second distal end, the single proximal portion of the back strap fastened to a back portion of the band, the first distal end fastened to a first back connector portion located on a back section of the receiving body and the second distal end fastened to a second back connector portion located on the back section of the receiving body; and
(b) a drainage element configured to extend a urine flow away from a user's body, the open outlet of the receiving body fastened to the drainage element, the drainage element in fluid communication with the receiving body.

17. The device according to claim 16 wherein the band is constructed from a stretchable material.

18. The device according to claim 17 wherein a user is a male.

19. The device according to claim 18 wherein the receiving body is constructed from polyurethane.

20. A kit comprising a reusable urine directional device configured to extend a urine flow away from a user's body; a urine disposal assembly; and a reusable wash bag configured to hold the urine directional device during a cleansing process.
